(19) United States
(12) Patent Application Publication
(10) Pub. No.: US 2022/0313642 A1
(43) Pub. Date: Oct. 6, 2022

(54) TYROSINE DERIVATIVES AND COMPOSITIONS COMPRISING THEM

(71) Applicant: Tyme, Inc., Bedminster, NJ (US)

(72) Inventor: Steven HOFFMAN, Mahwah, NJ (US)

(21) Appl. No.: 17/847,951

(22) Filed: Jun. 23, 2022

Related U.S. Application Data
(63) Continuation of application No. 15/365,120, filed on Nov. 30, 2016.
Application No. 15/365,120 is a continuation-in-part of application No. 14/062,194, filed on Oct. 24, 2013, now Pat. No. 9,585,841.
(60) Provisional application No. 61/894,279, filed on Oct. 22, 2013.

(51) Int. Cl. A61K 31/198; A61K 45/06; A61K 33/40; A61K 47/55

(52) U.S. Cl. CPC A61K 31/198; A61K 45/06; A61K 33/40; C12Y 107/03003; C12Y 305/01001; A61K 47/55; A61K 38/00

(57) ABSTRACT

Compositions and kits comprising a tyrosine hydroxylase inhibitor and an anticancer agent that is chemically bonded to, or physically associated with, the tyrosine hydroxylase inhibitor are provided. Also provided are methods for reducing cell proliferation in a subject comprising administering to a subject in need thereof a composition comprising a tyrosine hydroxylase inhibitor and an anticancer agent that is chemically bonded to, or physically associated with, the tyrosine hydroxylase inhibitor.

### CROSS REFERENCE TO RELATED APPLICATIONS

This application is a continuation-in-part of U.S. patent application Ser. No. 14/062,194 filed on Oct. 24, 2013, which claims priority to U.S. Provisional Application Ser. No. 61/894,279 filed on Oct. 22, 2013. Both of these applications are incorporated herein by reference in their entirety.

### TECHNICAL FIELD

The present inventions relate generally to compositions, kits and methods for the reduction of cellular proliferation as, for example, in the treatment of cancer.

### BACKGROUND

According to the U.S. National Cancer Institute's Surveillance Epidemiology and End Results (SEER) database for the year 2008, the most recent year for which incidence data are available, 11,958,000 Americans have invasive cancers. Cancer is the second most common cause of death in the United States, behind only heart disease, and accounts for one in four deaths. It has been estimated that approximately 1600 Americans die of cancer each day. In addition to the medical, emotional and psychological costs of cancer, cancer has significant financial costs to both the individual and society. It is estimated by the National Institutes of Health that the overall costs of cancer in 2010 was $263.8 billion. In addition, it is estimated that another $140.1 billion is lost in productivity due to premature death.

Cancer treatments today include surgery, hormone therapy, radiation, chemotherapy, immunotherapy, targeted therapy, and combinations thereof. Surgical removal of cancer has advanced significantly; however, there remains a high chance of recurrence of the disease. Hormone therapy using drugs such as aromatase inhibitors and luteinizing hormone-releasing hormone analogs and inhibitors has been relatively effective in treating prostate and breast cancers. Radiation and the related techniques of conformal proton beam radiation therapy, stereotactic radiosurgery, stereotactic radiation therapy, intraoperative radiation therapy, chemical modifiers, and radio sensitizers are effective at killing cancerous cells, but can also kill and alter surrounding normal tissue. Chemotherapy drugs such as aminopterin, cisplatin, methotrexate, doxorubicin, daunorubicin and others alone and in combinations are effective at killing cancer cells, often by altering the DNA replication process. Biological response modifier (BRM) therapy, biologic therapy, biotherapy, or immunotherapy alter cancer cell growth or influence the natural immune response, and involve administering biologic agents to a patient such as an interferons, interleukins, and other cytokines and antibodies such as rituximab and trastuzumab and even cancer vaccines such as Sipuleucel-T.

Cancer treatment using chemotherapy is often hindered by dose-limiting side effects. Such side effects often result from the action of the chemotherapeutic agent on non-cancerous cells. This limitation of chemotherapy has led to development of targeted therapies and site-directed chemotherapy.

Recently, new targeted therapies have been developed to fight cancer. These targeted therapies differ from chemotherapy because chemotherapy works by killing both cancerous and normal cells, with greater effects on the cancerous cells. Targeted therapies work by influencing the processes that control growth, division, and the spread of cancer cells and signals that cause cancer cells to die naturally. One type of targeted therapy includes growth signal inhibitors such as trastuzumab, gefitinib, imatinib, centuximab, dasatinib and nilotinib. Another type of targeted therapy includes angiogenesis inhibitors such as bevacizumab that inhibit cancers from increasing surrounding vasculature and blood supply. A final type of targeted therapy includes apoptosis-inducing drugs that are able to induce direct cancer cell death.

Site-directed chemotherapy directs anticancer agents preferentially to cancer cells by means of a targeting molecule. The targeting molecule has a specific affinity for the cancer being treated. Antibodies are examples of targeting molecules that can be used to direct anticancer agents to specific cancer types. An antibody may recognize an antigen that is expressed on the surface of a specific type of cancer cell. By attaching anticancer agents to the antibody, the anticancer agents can be brought specifically to the cancer cells that are being targeted.

Although all of these treatments have been effective to one degree or another, they all have drawbacks and limitations. In addition to many of the treatments being expensive, they also are often too imprecise or the cancers are able to adapt to them and become resistant.

Thus, there is a great need for additional cancer treatments. In particular, there is a need for treatments for cancers that have become resistant to other forms of treatment.

### SUMMARY

The present invention provides compositions, kits, and methods for reducing undue cellular proliferation, including that associated with the treatment of cancer, and combination therapies. In certain embodiments, the invention provides compositions comprising a tyrosine hydroxylase inhibitor and an anticancer agent that is chemically bonded to, or physically associated with, the tyrosine hydroxylase inhibitor. Other embodiments provide methods of reducing cell proliferation and/or methods of treating cancer comprising administering an effective amount of such compositions. Some embodiments also provide combination therapies that are administered in conjunction with other therapeutic agents. In other embodiments, the invention provides kits that comprise such compositions together with suitable packaging.

### DETAILED DESCRIPTION OF ILLUSTRATIVE EMBODIMENTS

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. This condition, disease or disorder can be cancer.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired result in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These pharmaceutically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The term “stereoisomers” refers to compounds that have identical chemical constitution, but differ as regards the arrangement of the atoms or groups in space.

The term “administering” means either directly administering a compound or composition of the present invention, or administering a prodrug, derivative or analog which will form an equivalent amount of the active compound or substance within the body.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

The term “inhibitor” as used herein includes compounds that inhibit the expression or activity of a protein, polypeptide or enzyme and does not necessarily mean complete inhibition of expression and/or activity. Rather, the inhibition includes inhibition of the expression and/or activity of a protein, polypeptide or enzyme to an extent, and for a time, sufficient to produce the desired effect.

The term “promoter” as used herein includes compounds that promote the expression or activity of a protein, polypeptide or enzyme and does not necessarily mean complete promotion of expression and/or activity. Rather, the promotion includes promotion of the expression and/or activity of a protein, polypeptide or enzyme to an extent, and for a time, sufficient to produce the desired effect.

“Chemically bonded” refers to the connection of two atoms by a chemical bond. A chemical bond is a bond resulting from the electronic interaction of one atom (which may be part of a molecule) with another atom (which may be part of a molecule). Chemical bonds may be covalent bonds or non-covalent bonds.

“Physically associated” refers to two molecules that are maintained in close proximity by means other than a chemical bond. One example of physically associated molecules is the impregnation of one molecule into a sample of the other molecule. Another example when one molecule is encapsulated by another molecule.

The term “linker” refers to a chemical moiety that allows two molecules to be indirectly chemically bonded, or indirectly physically associated. A linker that indirectly chemically bonds two molecules forms separate chemical bonds with each of the two molecules such that the two molecules are connected through the linker. A linker that indirectly physically associates two molecules forms separate physically associations with each of the two molecules such that the two molecules are physically associated through the linker. A linker can also connect two molecules by chemically bonding to one of the two molecules and physically associating with the other of the two molecule.

In one embodiment, the present invention provides a chemotherapy that specifically directs anticancer agents to cancer cells. While not intending to be bound by any particular mechanism of operation, the compositions of the present invention specifically target cancer cells and thereby provide a site-directed chemotherapy. It is believed that the tyrosine hydroxylase portion of the compositions of the present invention is absorbed by cancer cells. By chemically bonding, or physically associating, the tyrosine hydroxylase inhibitor and an anticancer agent, the anticancer agent accompanies the tyrosine hydroxylase inhibitor to, and/or into, the cancer cells. In this manner, the anticancer agent is directed to cancer cells in preference to non-cancerous cells.

The present invention provides compositions comprising a tyrosine hydroxylase inhibitor and an anticancer agent that is chemically bonded to, or physically associated with, the tyrosine hydroxylase inhibitor.

Representative tyrosine hydroxylase inhibitors that may be used in the compositions of the present invention include tyrosine derivatives, which typically are rapidly absorbed by most cancers and inflamed tissues. Thus, in some embodiments, the tyrosine hydroxylase inhibitor is a tyrosine derivative. Representative tyrosine derivatives that may be used in compositions of the present invention include one or more of methyl (2R)-2-amino-3-(2-chloro-4-hydroxyphenyl) propanoate, D-tyrosine ethyl ester hydrochloride, methyl (2R)-2-amino-3-(2,6-dichloro-3,4-dimethoxyphenyl) propanoate, H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OME HCl, H-D-3,5-diiodo-tyr-OME HCl, D-tyrosine methyl ester hydrochloride, D-tyrosine-OMe HCl, methyl D-tyrosinate hydrochloride, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)—OSu, Fmoc-tyr(3-NO₂)—OH, α-methyl-L-tyrosine, α-methyl-D-tyrosine, and α-methyl-DL-tyrosine (also known as DL-2-Methyl-3-(4-Hydroxyphenyl) alanine). These tyrosine derivatives are referred to herein as the “representative tyrosine derivatives.” In some embodiments, the tyrosine derivative is α-methyl-DL-tyrosine.

Anticancer agents that may be used in compositions of the present invention include any agents that are active against cancer, and include alkylating agents, antimetabolites, anti-microtubule agents, topoisomerase inhibitors, cytotoxic antibiotics, selective estrogen receptor modulators, aromatase inhibitors, signal transduction inhibitors, agents that modify the function of proteins that regulate gene expression and other cellular functions, drugs that induce cancer cells to undergo apoptosis, and drugs that interfere with angiogenesis.

Representative anticancer agents that may be used in the present invention include 5-fluorouracil, abiraterone acetate, acetylcholine, ado-trastuzumab emtansine, afatinib, aldesleukin, alectinib, alemtuzumab, alitretinoin, aminolevulinic acid, anastrozole, anastrozole, aprepitant, arsenic trioxide, asparaginase erwinia chrysanthemi, atezolizumab, axitinib, azacitidine, belinostat, bendamustine, benzyl isothiocyanate, bevacizumab, bexarotene, bicalutamide, bleomycin, blinatumomab, bortezomib, bosutinib, brentuximab vedotin, busulfan, cabazitaxel, cabozantinib, capecitabine, carboplatin, carfilzomib, carmustine, ceritinib, cetuximab, chlorambucil, cisplatin, clofarabine, cobimetinib, crizotinib, cyclophosphamide, cytarabine, dabrafenib, dacarbazine, dacarbazine, dactinomycin, daratumumab, dasatinib, daunorubicin, decitabine, defibrotide sodium, degarelix, denileukin diftitox, denosumab, dexamethasone, dexrazoxane, dihydrotestosterone (DHT), dinutuximab, docetaxel, doxorubicin, elotuzumab, eltrombopag, enzalutamide, epirubicin, eribulin mesylate, erlotinib, etoposide, everolimus, exemestane, exemestane, filgrastim, fludarabine phosphate, flutamide, fulvestrant, fulvestrant, gefitinib, gemcitabine, gemtuzumab, gemtuzumab ozogamicin, glucarpidase, goserelin acetate, hydroxyurea, ibritumomab tiuxetan, ibrutinib, idarubicin, idelalisib, ifosfamide, imatinib, imiquimod, interferon alfa-2b, ipilimumab, irinotecan, ixabepilone, ixazomib, lanreotide, lapatinib, lenalidomide, lenvatinib, letrozole, leucovorin, leuprolide, lomustine, mechlorethamine, megestrol acetate, melphalan, mercaptopurine, mesna, methotrexate, mitomycin C, mitoxantrone, necitumumab, nelarabine, netupitant, nilotinib, nilutamide, nivolumab, obinutuzumab, ofatumumab, olaparib, omacetaxine mepesuccinate, osimertinib, oxaliplatin, ozogamicin, paclitaxel, palbociclib, palifermin, pamidronate, panitumumab, panobinostat, pazopanib, pegaspargase, peginterferon alfa-2b, pembrolizumab, pemetrexed, pertuzumab, plerixafor, pomalidomide, ponatinib, pralatrexate, prednisone, procarbazine, propranolol, radium 223 dichloride, raloxifene, ramucirumab, rasburicase, regorafenib, rituximab, rolapitant, romidepsin, romiplostim, ruxolitinib, siltuximab, sipuleucel-t, sonidegib, sorafenib, sunitinib, talimogene laherparepvec, tamoxifen, temozolomide, temsirolimus, thalidomide, thioguanine, thiotepa, tipiracil, topotecan, toremifene, toremifene, tositumomab, trabectedin, trametinib, trastuzumab, tretinoin, trifluridine, uridine triacetate, vandetanib, vemurafenib, venetoclax, vinblastine, vincristine, vinorelbine, vismodegib, vorinostat, ziv-aflibercept, zoledronic acid, and pharmaceutically acceptable salts thereof. These anticancer agents are referred to herein as the “representative anticancer agents.”

In some embodiments, the anticancer agent is one or more of 5-fluorouracil, capecitabine, cisplatin, erlotinib, everolimus, gemcitabine, irinotecan, lanreotide acetate, leucovorin, mitomycin C, oxaliplatin, paclitaxel, taxotere, and sunitinib malate.

In some embodiments of the present invention, the anticancer agent is chemically bonded to the tyrosine hydroxylase inhibitor. The anticancer agent can be chemically bonded to the tyrosine hydroxylase inhibitor by a covalent bond. A covalent bond between the tyrosine hydroxylase inhibitor and the anticancer agent may be formed by chemical reaction of reactive functional groups on the tyrosine hydroxylase inhibitor with reactive functional groups on the anticancer agent. Reactive function groups on the tyrosine hydroxylase inhibitor may include esters, carboxylic acids, amides, amino groups, hydroxyl groups, and activated aromatic or aliphatic carbon atoms. Reactive function groups on the anticancer agent may include esters, carboxylic acids, amides, amino groups, hydroxyl groups, activated aromatic or aliphatic carbon atoms, sulfides, and cyano groups. Thus, reaction of functional groups on the tyrosine hydroxylase inhibitor with functional groups on the anticancer agent can result in ethers, amines, esters, amides, thioesters, thioethers, carbamates, and ureas. Methods for making the types of covalent bonds required by embodiments of the present invention are generally known in the art. See, e.g., Michael B. Smith and Jerry March, March's Advanced Organic Chemistry, Reactions, Mechanism, and Structure (John Wiley & Sons 2001).

An exemplary embodiment of the present invention wherein an anticancer agent is chemically bonded to a tyrosine hydroxylase inhibitor by a covalent bond is the ester that would result from condensing a hydroxyl group of capecitabine with the carboxylic acid group of α-methyl tyrosine:

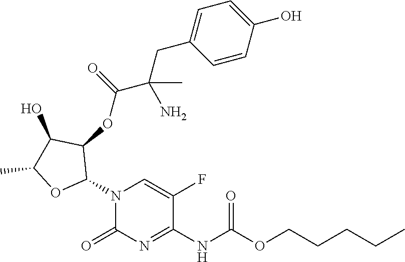 [SMILES: CCCCCOC(=O)Nc1nc(=O)n([C@@H]2O[C@H](C)[C@@H](O)[C@H]2OC(=O)C(C)(N)Cc2ccc(O)cc2)cc1F]

In other embodiments the anticancer agent is chemically bonded to the tyrosine hydroxylase inhibitor by a non-covalent bond. In some embodiments, the non-covalent bond is an ionic bond. An ionic bond between the tyrosine hydroxylase inhibitor and the anticancer agent may be formed by chemical reaction of salt-forming functional groups on the tyrosine hydroxylase inhibitor with salt-forming functional groups on the anticancer agent. Salt-forming functional groups on the tyrosine hydroxylase inhibitor may include carboxylic acids, amino groups, and acidic hydroxyl groups. Salt-forming functional groups on the anticancer agent may include carboxylic acids, amino groups, acidic hydroxyl groups, and acidic thiols. Methods for making the types of non-covalent bonds required by embodiments of the present invention are generally known in the art. See, e.g., Michael B. Smith and Jerry March, March's Advanced Organic Chemistry, Reactions, Mechanism, and Structure (John Wiley & Sons 2001).

An exemplary embodiment of the present invention wherein an anticancer agent is chemically bonded to a tyrosine hydroxylase inhibitor by a non-covalent, ionic bond is the salt that would result from reacting D-tyrosine ethyl ester with leucovorin:

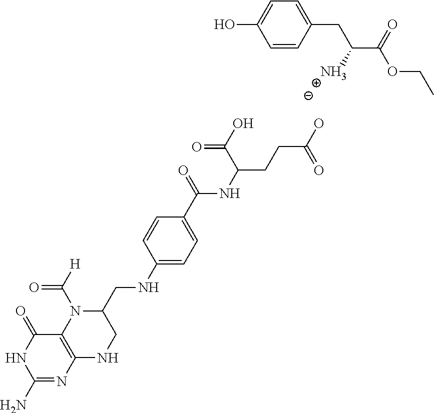 [SMILES: CCOC(=O)[C@H]([NH3+])Cc1ccc(O)cc1 ; Nc1nc2c(c(=O)[nH]1)N(C=O)C(CNc1ccc(C(=O)NC(CCC(=O)O)C(=O)O)cc1)CN2]

In other embodiments of the present invention, the anticancer agent is chemically bonded to the tyrosine hydroxylase inhibitor through a linker. In some embodiments, the linker is a chemical moiety that forms chemical bonds to both the tyrosine hydroxylase inhibitor and the anticancer agent and thus separates the tyrosine hydroxylase inhibitor and the anticancer agent. Thus, in some embodiments, the present invention provides a tyrosine hydroxylase inhibitor-anticancer agent conjugate. Covalent bonds between the tyrosine hydroxylase inhibitor and the linker may include, for example, carbamate bonds, amide bonds, ester bonds, amino bonds, and ether bonds formed by reaction of reactive functional groups on the tyrosine hydroxylase inhibitor with reactive functional groups on the linker. Covalent bonds between the anticancer agent and the linker may include, for example, disulfide bonds, carbamate bonds, amide bonds, ester bonds, amino bonds, and ether bonds formed by reaction of reactive functional groups on the anticancer agent with reactive functional groups on the linker. Thus, in some embodiments, the linker is a chemical moiety having reactive functional groups that react with the tyrosine hydroxylase inhibitor, and reactive functional groups that react with the anticancer agent. In some embodiments, the linker is a molecule with two functional groups, one of which is capable of forming a covalent bond with a functional group on the tyrosine hydroxylase inhibitor, and the other of which is capable of forming a covalent bond with a functional group on the anticancer agent. In some embodiments, the linker is selected from aliphatic compounds having two or more reactive functional groups, aromatic compounds having two or more reactive functional groups, carbohydrates, amino acids, peptides, diamino compounds, polyamino compounds, diols, polyols, amino-alcohols, ethanolamine, diamides, polyamides, lipids, and polyethylene glycols. Methods for making the chemical bonds required for embodiments of the present invention are generally known in the art. See, e.g., Michael B. Smith and Jerry March, March's Advanced Organic Chemistry, Reactions, Mechanism, and Structure (John Wiley & Sons 2001).

In some embodiments, the linker can be cleaved under physiological conditions, thereby disjoining the tyrosine hydroxylase inhibitor and the anticancer agent. In some embodiments, the linker is cleaved when the conjugate has been taken into the cancer cell. In some embodiments, the linker is cleaved by enzymes located within, or on the surface of, the cancer cell.

In some embodiments, a single linker may be chemically bound to multiple anticancer agents and to a single tyrosine hydroxylase inhibitor.

In other embodiments of the present invention, the anticancer agent is physically associated with the tyrosine hydroxylase inhibitor. In some embodiments, the anticancer agent is physically associated with the tyrosine hydroxylase inhibitor by impregnation. Impregnation may be achieved by and applying force to a tyrosine hydroxylase inhibitor and a solid anticancer agent for a time and under conditions effective to impregnate at least one of the tyrosine hydroxylase inhibitor and the anticancer agent with the other of said tyrosine hydroxylase inhibitor and said anticancer agent. Compositions formed by impregnation and methods of impregnation are set forth in U.S. Patent Application Publication No. 2015/0112116-A1, published on Apr. 23, 2015, the contents of which are incorporated in their entirety herein.

In other embodiments, the anticancer agent is physically associated with the tyrosine hydroxylase inhibitor by encapsulation.

In other embodiments, the linker joins the tyrosine hydroxylase inhibitor to the anticancer agent through physical association, or through a combination of physical association and chemical bonding. In some embodiments, the anticancer agent is encapsulated within a liposome that has one or more tyrosine hydroxylase inhibitors covalently bonded to its outer surface. In this embodiment, the liposome is a linker that bonds to the tyrosine hydroxylase inhibitor by chemical (covalent) bonds, and binds to the anticancer agent by physical association (encapsulation).

In some embodiments of the present invention, the compositions of the present invention further comprise one or more pharmaceutically acceptable excipients. Pharmaceutically acceptable excipients are known in the art. See, e.g., Remington's Pharmaceutical Sciences, 18^{th} Edition, Mack Publishing Company (1990).

Methods of treating cancer in a subject also are provided, as are methods of reducing undue cellular proliferation. Such methods can include administering an effective amount of a composition that targets cancer cells. Suitable embodiments are methods that include administering an effective amount of the above-noted composition comprising a tyrosine hydroxylase inhibitor and an anticancer agent that is chemically bonded to, or physically associated with, the tyrosine hydroxylase inhibitor. Other suitable methods include administering an effective amount of the above-noted composition comprising a tyrosine hydroxylase inhibitor and an anticancer agent that is chemically bonded to, or physically associated with, the tyrosine hydroxylase inhibitor, in conjunction with one or more additional therapeutic agents.

The compositions, with or without additional therapeutic agents, may be provided in a single dosage form or any number of desired dosage forms, including in individual dosage forms. Representative dosage forms include tablets, capsules, caplets, sterile aqueous or organic solutions, reconstitutable powders, elixirs, liquids, colloidal or other types of suspensions, emulsions, beads, beadlets, granules, microparticles, nanoparticles, and combinations thereof. The amount of composition administered will, of course, be dependent on the subject being treated, the subject's weight, the severity of the condition being treated, the manner of administration, and the judgment of the prescribing physician.

Administration of the compositions, with or without additional therapeutic agents, can be through various routes, including orally, nasally, subcutaneously, intravenously, intramuscularly, transdermally, vaginally, rectally or in any combination thereof. Transdermal administration can be effected using, for example, oleic acid, 1-methyl-2-pyrrolidone, or dodecylnonaoxyethylene glycol monoether.

The subject to which the instant compositions, with or without additional therapeutic agents, are administered can be a mammal, preferably a human.

Representative methods include those in which the cancer is non-small cell lung cancer. In certain embodiments, the non-small cell lung cancer is stage IV non-small cell lung cancer. In yet other embodiments, the cancer is ovarian cancer, breast cancer, cervical cancer, pancreatic cancer, stomach cancer, brain cancer, liver cancer, testicular cancer, leukemia, lymphoma, appendix cancer, biliary cancer, choleangiocarcinoma, colon cancer, colorectal cancer, germ cell tumor, glioma, Hodgkin's lymphoma, lung cancer, neuroblastoma, prostate cancer, renal cancer, sarcoma, thyroid cancer, tongue cancer, tonsil squamous cell carcinoma, or urothelial cancer. In some embodiments, the cancer is pancreatic cancer.

The present methods can include not only the disclosed administration step but also the step of assessing progression of said cancer in said subject and/or the extent of cellular proliferation. The assessing step can be performed before or after the administering step.

Suitable embodiments can include administering the above-noted compositions comprising a tyrosine hydroxylase inhibitor and an anticancer agent that is chemically bonded to, or physically associated with, the tyrosine hydroxylase inhibitor. The tyrosine hydroxylase inhibitor in the composition can be a tyrosine derivative. The tyrosine derivative may be one or more of the representative tyrosine derivatives noted above.

The anticancer agents in the above-noted compositions include any agents that are active against cancer, and include alkylating agents, antimetabolites, anti-microtubule agents, topoisomerase inhibitors, cytotoxic antibiotics, selective estrogen receptor modulators, aromatase inhibitors, signal transduction inhibitors, agents that modify the function of proteins that regulate gene expression and other cellular functions, drugs that induce cancer cells to undergo apoptosis, and drugs that interfere with angiogenesis. The anticancer agent may be one or more of the representative anticancer agents noted above.

Methods of treating cancer in a subject are also provided comprising administering to the subject in need thereof an effective amount of the above-noted composition comprising a tyrosine hydroxylase inhibitor and an anticancer agent that is chemically bonded to, or physically associated with, the tyrosine hydroxylase inhibitor. In suitable embodiments, the composition is administered orally, subcutaneously, intravenously, transdermally, vaginally, rectally or in any combination thereof. The transdermal administration can be done with oleic acid, 1-methyl-2-pyrrolidone, or dodecylnonaoxyethylene glycol monoether. In other embodiments, the composition is administered during a cycle consisting of five to seven days of administering the composition and one to two days of not administering the composition. The composition can be administered over the course of at least six of said cycles. The tyrosine hydroxylase inhibitor in the composition for treating cancer can be a tyrosine derivative. The tyrosine derivative may be one or more of the representative tyrosine derivatives noted above.

The anticancer agents that may be used in compositions for treating cancer include any agents that are active against cancer, and include alkylating agents, antimetabolites, anti-microtubule agents, topoisomerase inhibitors, cytotoxic antibiotics, selective estrogen receptor modulators, aromatase inhibitors, signal transduction inhibitors, agents that modify the function of proteins that regulate gene expression and other cellular functions, drugs that induce cancer cells to undergo apoptosis, and drugs that interfere with angiogenesis. The anticancer agent may be one or more of the representative anticancer agents noted above.

The subject in methods of treating cancer can be a mammal and that mammal can be a human. In some embodiments, the subject is a human.

Representative methods of treating cancer include those in which the cancer is non-small cell lung cancer. In certain embodiments, the non-small cell lung cancer is stage IV non-small cell lung cancer. In other embodiments, the cancer is ovarian cancer, breast cancer, cervical cancer, pancreatic cancer, stomach cancer, brain cancer, liver cancer, testicular cancer, leukemia, lymphoma, appendix cancer, biliary cancer, choleangiocarcinoma, colon cancer, colorectal cancer, germ cell tumor, glioma, Hodgkin's lymphoma, lung cancer, neuroblastoma, prostate cancer, renal cancer, sarcoma, thyroid cancer, tongue cancer, tonsil squamous cell carcinoma, or urothelial cancer. In some embodiments, the cancer is pancreatic cancer.

Another suitable embodiment further comprises assessing progression of said cancer in said subject. The assessing step can be performed before said administering step or the assessing step can be performed after said administering step.

In some embodiments, the methods of treating cancer further comprise administering one or more additional therapeutic agents. Such additional therapeutic agents may include anticancer agents which are the same as, or different than, the anticancer agents that are components of the above-noted compositions. In some embodiments, the additional therapeutic agents are one or more of the representative anticancer agents noted above.

In other embodiments, the additional therapeutic agents may include one or more additional tyrosine hydroxylase inhibitors, melanin and/or a melanin promoter, a p450 3A4 promoter, a leucine aminopeptidase inhibitor, and a growth hormone inhibitor. In some embodiments, at least two of the additional therapeutic agents (i.e., melanin, promoters and/or inhibitors) are administered simultaneously. In other embodiments, at least three of the additional therapeutic agents are administered simultaneously. Each of the additional therapeutic agents can be administered simultaneously.

In some embodiments, the additional therapeutic agent is one or more tyrosine hydroxylase inhibitors. Representative tyrosine hydroxylase inhibitors include tyrosine derivatives. The tyrosine derivative may be one or more of the representative tyrosine derivatives noted above.

In some embodiments, the additional therapeutic agents include at least one of melanin, a melanin promoter, or a combination thereof. Thus, melanin can be used, one or more melanin promoters can be used, and both melanin and one or more melanin promoters can be used (either in separate dosage forms or in the same dosage form). Melanin promoters according to the present invention are chemical compounds that increase the production and/or the activity of melanin. Increased melanin levels are believed to reduce inflammation (through, for example, suppression of TNF) and exclude the sequestered lymph system. Melanin is a photo catalyst, and can therefore promote chemical reactions that generate free radicals which, in turn, can become accessible to cancer cells. Representative melanin promoters are methoxsalen and melanotan II.

In some embodiments, the additional therapeutic agents include a p450 3A4 promoter. “Cytochrome p450 3A4” (which can be abbreviated as “p450 3A4”) is a member of the cytochrome p450 superfamily of enzymes, and is a mixed-function oxidase that is involved in the metabolism of xenobiotics in the body. It has the widest range of substrates of all of the cytochromes. Representative p450 3A4 promoters are 5,5-diphenylhydantoin (sold commercially as, for example, Dilantin), valproic acid, and carbamazepine, which are believed to induce expression of the p450 3A4 enzyme.

In some embodiments, the additional therapeutic agents include leucine aminopeptidase inhibitors (alternatively known as leucyl aminopeptidase inhibitors). Leucine aminopeptidases are enzymes that preferentially catalyze the hydrolysis of leucine residues at the N-terminus of peptides and/or proteins. Representative leucine aminopeptidase inhibitors are N-[(2S,3R)-3-amino-2-hydroxy-4-phenylbutyryl]-L-leucine, and rapamycin.

In some embodiments, the additional therapeutic agents include a growth hormone inhibitor. Growth hormone (such as, for example, pancreatic growth hormone) induces cell replication. Representative growth hormone inhibitors are octreotide, somatostatin, and seglitide.

In some embodiments, the additional therapeutic agents include D-leucine. D-leucine is a stereoisomer of the naturally occurring L-leucine, the form of leucine incorporated into polypeptides and proteins.

Methods of reducing cell proliferation in a subject are also provided comprising administering an effective amount of a composition comprising a tyrosine hydroxylase inhibitor and an anticancer agent that is chemically bonded to, or physically associated with, the tyrosine hydroxylase inhibitor. In suitable embodiments, components are administered orally, subcutaneously, intravenously, transdermally, vaginally, rectally or in any combination thereof. The transdermal administration can be done with oleic acid, 1-methyl-2-pyrrolidone, or dodecylnonaoxyethylene glycol monoether. In other embodiments, the composition is administered during a cycle consisting of five to seven days of administering the composition and one to two days of not administering the composition. The composition can be administered over the course of at least six of said cycles. The tyrosine hydroxylase inhibitor in the composition for reducing cell proliferation can be a tyrosine derivative. The tyrosine derivative may be one or more of the representative tyrosine derivatives noted above.

The anticancer agents that may be used in compositions for reducing cell proliferation include any agents that are active against cancer, and include alkylating agents, antimetabolites, anti-microtubule agents, topoisomerase inhibitors, cytotoxic antibiotics, selective estrogen receptor modulators, aromatase inhibitors, signal transduction inhibitors, agents that modify the function of proteins that regulate gene expression and other cellular functions, drugs that induce cancer cells to undergo apoptosis, and drugs that interfere with angiogenesis. The anticancer agent may be one or more of the representative anticancer agents noted above.

The subject in methods of reducing cell proliferation can be a mammal and the mammal can be a human. In some embodiments, the subject is a human.

Representative methods of reducing cell proliferation include those in which the cell proliferation is cancer. In some embodiments, the cancer is non-small cell lung cancer. In certain embodiments, the non-small cell lung cancer is stage IV non-small cell lung cancer. In other embodiments, the cancer is ovarian cancer, breast cancer, cervical cancer, pancreatic cancer, stomach cancer, brain cancer, liver cancer, testicular cancer, leukemia, lymphoma, appendix cancer, biliary cancer, choleangiocarcinoma, colon cancer, colorectal cancer, germ cell tumor, glioma, Hodgkin's lymphoma, lung cancer, neuroblastoma, prostate cancer, renal cancer, sarcoma, thyroid cancer, tongue cancer, tonsil squamous cell carcinoma, or urothelial cancer. In some embodiments, the cancer is pancreatic cancer.

Another suitable embodiment further comprises assessing progression of said cancer in said subject. The assessing step can be performed before said administering step or the assessing step can be performed after said administering step.

In some embodiments, the methods of reducing cell proliferation further comprise administering one or more additional therapeutic agents. Such additional therapeutic agents may include anticancer agents which are the same as, or different than, the anticancer agents that are components of the above-noted compositions. In some embodiments, the additional therapeutic agents are one or more of the representative anticancer agents noted above.

In other embodiments, the additional therapeutic agents may include one or more additional tyrosine hydroxylase inhibitors, melanin and/or a melanin promoter, a p450 3A4 promoter, a leucine aminopeptidase inhibitor, and a growth hormone inhibitor. In some embodiments, at least two of the additional therapeutic agents (i.e., melanin, promoters and/or inhibitors) are administered simultaneously. In other embodiments, at least three of the additional therapeutic agents are administered simultaneously. Each of the additional therapeutic agents can be administered simultaneously.

In some embodiments, the additional therapeutic agent is one or more tyrosine hydroxylase inhibitors. Representative tyrosine hydroxylase inhibitors include tyrosine derivatives. The tyrosine derivative may be one or more of the representative tyrosine derivatives noted above.

In some embodiments, the additional therapeutic agents include at least one of melanin, a melanin promoter, or a combination thereof. Thus, melanin can be used, one or more melanin promoters can be used, and both melanin and one or more melanin promoters can be used (either in separate dosage forms or in the same dosage form). Melanin promoters according to the present invention are chemical compounds that increase the production and/or the activity of melanin. Increased melanin levels are believed to reduce inflammation (through, for example, suppression of TNF) and exclude the sequestered lymph system. Melanin is a photo catalyst, and can therefore promote chemical reactions that generate free radicals which, in turn, can become accessible to cancer cells. Representative melanin promoters are methoxsalen and melanotan II.

In some embodiments, the additional therapeutic agents include a p450 3A4 promoter. “Cytochrome p450 3A4” (which can be abbreviated as “p450 3A4”) is a member of the cytochrome p450 superfamily of enzymes, and is a mixed-function oxidase that is involved in the metabolism of xenobiotics in the body. It has the widest range of substrates of all of the cytochromes. Representative p450 3A4 promoters are 5,5-diphenylhydantoin (sold commercially as, for example, Dilantin), valproic acid, and carbamazepine, which are believed to induce expression of the p450 3A4 enzyme.

In some embodiments, the additional therapeutic agents include leucine aminopeptidase inhibitors (alternatively known as leucyl aminopeptidase inhibitors). Leucine aminopeptidases are enzymes that preferentially catalyze the hydrolysis of leucine residues at the N-terminus of peptides and/or proteins. Representative leucine aminopeptidase inhibitors are N-[(2S,3R)-3-amino-2-hydroxy-4-phenylbutyryl]-L-leucine, and rapamycin.

In some embodiments, the additional therapeutic agents include a growth hormone inhibitor. Growth hormone (such as, for example, pancreatic growth hormone) induces cell replication. Representative growth hormone inhibitors are octreotide, somatostatin, and seglitide.

In some embodiments, the additional therapeutic agents include D-leucine. D-leucine is a stereoisomer of the naturally occurring L-leucine, the form of leucine incorporated into polypeptides and proteins.

Also provided herein are kits including a therapy that specifically targets cancer cells. Representative kits comprise a composition comprising a tyrosine hydroxylase inhibitor and an anticancer agent that is chemically bonded to, or physically associated with, said tyrosine hydroxylase inhibitor together with suitable packaging for same. The kit can include one or more separate containers, dividers or compartments and, optionally, informational material such as instructions for administration. For example, each composition can be contained in a bottle, vial, or syringe, and the informational material can be contained in a plastic sleeve or packet or provided in a label. In some embodiments, the kit includes a plurality (e.g., a pack) of individual containers, each containing one or more unit dosage forms of a composition described herein. For example, the kit can include a plurality of syringes, ampules, foil packets, or blister packs, each containing a single unit dose of a composition described herein or any of the various combinations thereof. The containers of the kits can be air tight, waterproof (e.g., impermeable to changes in moisture or evaporation), and/or light-tight. The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In some embodiments, the kit further comprises one or more additional therapeutic agents. In some embodiments, the additional therapeutic agents are selected from anticancer agents which are the same as, or different than, the anticancer agents that are components of the above-noted compositions, additional tyrosine hydroxylase inhibitors, melanin, a melanin promoter, a p450 3A4 promoter, leucine aminopeptidase inhibitors, a growth hormone inhibitor, and D-leucine.

The following example is offered for illustrative purposes only, and is not intended to limit the scope of the present invention in any way.

### EXAMPLE 1

The ability of the tyrosine hydroxylase portion of the above-noted compositions to be taken into pancreas cells is demonstrated by the following study.

Background: SM88 is a novel combination of five therapies (sirolimus, melanin, melanotan, phenytoin, and tyrosine isomers) that when administered together, has demonstrated anti-cancer activity with little to no toxicity in a preliminary study of 30 patients [J Clin Oncol 31, 2013 (suppl; abstr e22095) and Hoffman et al. Gyn Oncol, 130(1), e43]. This study reports preclinical animal data related to toxicity of the tyrosine agent, α-methyl-DL-tyrosine, and possible mechanism of action.

Material and Methods: Preclinical animal model with 7 day escalating dose and 28 day repeat dosing toxicology studies in Sprague Dawley rats and beagle dogs using α-methyl-DL-tyrosine. Test and control/vehicle items were administered daily or three times per week over a 4-week period at dose levels of 25, 75, 150, and 300 mg/kg. The study included mortality, clinical observations, body weights, food consumption, electrocardiography and ophthalmology. In addition, hematology, coagulation, clinical chemistry and urinalysis parameters were evaluated during pretreatment as well as on Day 29 (Main and Recovery animals) and on Day 55 (Recovery animals). Blood samples were collected on Days 1 and 27 at 8 time points relative to treatment in order to determine the toxicokinetic profile.

Results: All test rats regardless of sex, demonstrated consistent organ volume decrease in the pancreas (decreased cell volume, and reduced concentration of zymogenous vacuoles), ovaries and uterus. These changes were completely reversible upon the discontinuation of α-methyl-DL-tyrosine. Twenty eight (28) consecutive days resulted in a reduction in mean body weight gain in the 300 mg/kg/day males, which correlated with a decrease in food consumption and a dose-related increase in mean body weight gain in the females of all dose groups. Dogs had no such observations. There were no deaths, no clinical signs, no effects on ECGs, no ocular findings, no changes in hematology, coagulation, clinical chemistry and urinalysis parameters, no changes in other organ weights and no macroscopic and microscopic findings that could be attributed to the administration of the Isomers at doses up to 300 mg/kg. Consequently the No Observed Effect Level (NOEL) for the tyrosine agent when administered three times per week for 4 weeks was determined to be 150 (for dog) and 300 (for rat) mg/kg. Plasma Cmax values at 150 mg/kg (dog) obtained on Day 27 were 41.7 μg/ml and 41.36 μg/ml for males and females respectively. AUC_{0-Tlast} values were 717.7 (males) and 724.8 (females) hr*μg/ml. The difference in combined isomer concentrations in plasma between Day 1 and 27, show that the systemic exposures to the agent generally increased dose-dependently, and in a slightly less than dose-proportional manner. In general, the agent's maximum concentration levels (Cmax) were reached at 2 to 6.7 hours post-dosing. After Tₘₐₓ, the agent's plasma concentrations declined gradually at a mean estimated T1/2 value ranging from 7.9 to 9.3 hours on Day 1 and from 8.4 to 9.6 hours on Day 27. There were no major or consistent sex-related differences as evidence by the sex ratios which ranged between 0.3 and 1.8 for all measured toxicokinetic parameters. Over the 4-week treatment period, AUC_{0-Tlast} and AUCINF (Cₘₐₓ) accumulation ratios (Day 27/Day 1) ranged from 0.6 to 1.8 (0.8 to 1.6) in the animals treated with 25, 75 and 150 mg/kg, suggesting that the agent does not accumulate when administered three (3) times per week over a 27 day period at doses up to 150 mg/kg. For rats, the results were similar.

What is claimed:

1. A composition comprising a tyrosine hydroxylase inhibitor and an anticancer agent that is chemically bonded to, or physically associated with, said tyrosine hydroxylase inhibitor.

2. The composition of claim 1 wherein the tyrosine hydroxylase inhibitor is a tyrosine derivative.

3. The composition of claim 2 wherein the tyrosine derivative is one or more of:
methyl (2R)-2-amino-3-(2-chloro-4-hydroxyphenyl) propanoate;
D-tyrosine ethyl ester hydrochloride;
methyl (2R)-2-amino-3-(2,6-dichloro-3,4-dimethoxyphenyl) propanoate;
H-D-Tyr(TBU)-allyl ester HCl;
methyl (2R)-2-amino-3-(3-chloro-4,5-dimethoxyphenyl) propanoate;
methyl (2R)-2-amino-3-(2-chloro-3-hydroxy-4-methoxyphenyl) propanoate;
methyl (2R)-2-amino-3-(4-[(2-chloro-6-fluorophenyl) methoxy] phenyl) propanoate;
methyl (2R)-2-amino-3-(2-chloro-3,4-dimethoxyphenyl) propanoate;
methyl (2R)-2-amino-3-(3-chloro-5-fluoro-4-hydroxyphenyl) propanoate;
diethyl 2-(acetylamino)-2-(4-[(2-chloro-6-fluorobenzyl) oxy] benzyl malonate;
methyl (2R)-2-amino-3-(3-chloro-4-methoxyphenyl) propanoate;
methyl (2R)-2-amino-3-(3-chloro-4-hydroxy-5-methoxyphenyl) propanoate;
methyl (2R)-2-amino-3-(2,6-dichloro-3-hydroxy-4-methoxyphenyl) propanoate;
methyl (2R)-2-amino-3-(3-chloro-4-hydroxyphenyl) propanoate;
H-DL-tyr-OMe HCl;
H-3,5-diiodo-tyr-OME HCl;
H-D-3,5-diiodo-tyr-OME HCl;
H-D-tyr-OMe HCl;
D-tyrosine methyl ester hydrochloride;
D-tyrosine-OMe HCl;
methyl D-tyrosinate hydrochloride;
D-tyrosine methyl ester HCl;
H-D-Tyr-OMe-HCl;
(2R)-2-amino-3-(4-hydroxyphenyl) propionic acid;
(2R)-2-amino-3-(4-hydroxyphenyl) methyl ester hydrochloride;
methyl (2R)-2-amino-3-(4-hydroxyphenyl) propanoate hydrochloride;
methyl (2R)-2-azanyl-3-(4-hydroxyphenyl) propanoate hydrochloride;
3-chloro-L-tyrosine;
3-nitro-L-tyrosine;
3-nitro-L-tyrosine ethyl ester hydrochloride;
DL-m-tyrosine;
DL-o-tyrosine;
Boc-Tyr (3,5-I2)-Su;
Fmoc-tyr(3-NO2)-H;
α-methyl-L-tyrosine;
α-methyl-D-tyrosine; and
α-methyl-DL-tyrosine.

4. The composition of claim 3 wherein the tyrosine derivative is α-methyl-DL-tyrosine.

5. The composition of claim 1 wherein the anticancer agent is at least one of an alkylating agent, an antimetabolite, an anti-microtubule agent, a topoisomerase inhibitor, a cytotoxic antibiotic, a selective estrogen receptor modulator, an aromatase inhibitor, a signal transduction inhibitor, an agent that modifies the function of proteins that regulate gene expression and other cellular functions, a drug that induces cancer cells to undergo apoptosis, and a drug that interferes with angiogenesis.

6. The composition of claim 1 wherein the anticancer agent is at least one of 5-fluorouracil, abiraterone acetate, acetylcholine, ado-trastuzumab emtansine, afatinib, aldesleukin, alectinib, alemtuzumab, alitretinoin, aminolevulinic acid, anastrozole, anastrozole, aprepitant, arsenic trioxide, asparaginase erwinia chrysanthemi, atezolizumab, axitinib, azacitidine, belinostat, bendamustine, benzyl isothiocyanate, bevacizumab, bexarotene, bicalutamide, bleomycin, blinatumomab, bortezomib, bosutinib, brentuximab vedotin, busulfan, cabazitaxel, cabozantinib, capecitabine, carboplatin, carfilzomib, carmustine, ceritinib, cetuximab, chlorambucil, cisplatin, clofarabine, cobimetinib, crizotinib, cyclophosphamide, cytarabine, dabrafenib, dacarbazine, dacarbazine, dactinomycin, daratumumab, dasatinib, daunorubicin, decitabine, defibrotide sodium, degarelix, denileukin diftitox, denosumab, dexamethasone, dexrazoxane, dihydrotestosterone (DHT), dinutuximab, docetaxel, doxorubicin, elotuzumab, eltrombopag, enzalutamide, epirubicin, eribulin mesylate, erlotinib, etoposide, everolimus, exemestane, exemestane, filgrastim, fludarabine phosphate, flutamide, fulvestrant, fulvestrant, gefitinib, gemcitabine, gemtuzumab, gemtuzumab ozogamicin, glucarpidase, goserelin acetate, hydroxyurea, ibritumomab tiuxetan, ibrutinib, idarubicin, idelalisib, ifosfamide, imatinib, imiquimod, interferon alfa-2b, ipilimumab, irinotecan, ixabepilone, ixazomib, lanreotide, lapatinib, lenalidomide, lenvatinib, letrozole, leucovorin, leuprolide, lomustine, mechlorethamine, megestrol acetate, melphalan, mercaptopurine, mesna, methotrexate, mitomycin c, mitoxantrone, necitumumab, nelarabine, netupitant, nilotinib, nilutamide, nivolumab, obinutuzumab, ofatumumab, olaparib, omacetaxine mepesuccinate, osimertinib, oxaliplatin, ozogamicin, paclitaxel, palbociclib, palifermin, pamidronate, panitumumab, panobinostat, pazopanib, pegaspargase, peginterferon alfa-2b, pembrolizumab, pemetrexed, pertuzumab, plerixafor, pomalidomide, ponatinib, pralatrexate, prednisone, procarbazine, propranolol, radium 223 dichloride, raloxifene, ramucirumab, rasburicase, regorafenib, rituximab, rolapitant, romidepsin, romiplostim, ruxolitinib, siltuximab, sipuleucel-t, sonidegib, sorafenib, sunitinib, talimogene laherparepvec, tamoxifen, temozolomide, temsirolimus, thalidomide, thioguanine, thiotepa, tipiracil, topotecan, toremifene, toremifene, tositumomab, trabectedin, trametinib, trastuzumab, tretinoin, trifluridine, uridine triacetate, vandetanib, vemurafenib, venetoclax, vinblastine, vincristine, vinorelbine, vismodegib, vorinostat, ziv-aflibercept, zoledronic acid, and pharmaceutically acceptable salts thereof

7. The composition of claim 1, wherein the anticancer agent is chemically bonded to the tyrosine hydroxylase inhibitor.

8. The composition of claim 7 wherein the anticancer agent is chemically bonded to the tyrosine hydroxylase inhibitor by a covalent bond.

9. The composition of claim 7, wherein the anticancer agent is chemically bonded to the tyrosine hydroxylase inhibitor by a non-covalent bond.

10. The composition of claim 7, wherein the anticancer agent is chemically bonded to the tyrosine hydroxylase inhibitor through a linker.

11. The composition of claim 1, wherein the anticancer agent is physically associated with the tyrosine hydroxylase inhibitor.

12. The composition of claim 11, wherein the anticancer agent is physically associated with the tyrosine hydroxylase inhibitor by impregnation.

13. The composition of claim 11, wherein the anticancer agent is physically associated with the tyrosine hydroxylase inhibitor by encapsulation.

14. The composition of claim 1 further comprising a pharmaceutically acceptable excipient.

15. A method of reducing cell proliferation in a subject comprising administering to the subject in need thereof an effective amount of the composition according to claim 1.

16. A method of treating cancer in a subject comprising administering to the subject in need thereof an effective amount of the composition according to claim 1.

17. The method of claim 16 wherein the cancer is at least one of non-small cell lung cancer, ovarian cancer, breast cancer, cervical cancer, pancreatic cancer, stomach cancer, brain cancer, liver cancer, testicular cancer, leukemia, lymphoma, appendix cancer, biliary cancer, choleangiocarcinoma, colon cancer, colorectal cancer, germ cell tumor, glioma, Hodgkin's lymphoma, lung cancer, neuroblastoma, prostate cancer, renal cancer, sarcoma, thyroid cancer, tongue cancer, tonsil squamous cell carcinoma, or urothelial cancer.

18. The method of claim 17 wherein the cancer is pancreatic cancer.

19. The method of claim 16 wherein the composition is administered orally, subcutaneously, intravenously, transdermally, vaginally, rectally or in any combination thereof

20. The method of claim 16, further comprising administering an effective amount of one or more additional therapeutic agents.

21. A kit comprising the composition according to claim 1 and suitable packaging.

22. The kit of claim 21, further comprising an additional therapeutic agent.
